(19) United States
(12) Patent Application Publication
(10) Pub. No.: US 2014/0004527 A1
(43) Pub. Date: Jan. 2, 2014

(54) DIAGNOSIS KIT AND METHOD OF USING THE SAME

(71) Applicant: PANASONIC CORPORATION, Osaka (JP)

(72) Inventors: Hiroaki OKA, Osaka (JP); Masaya NAKATANI, Hyogo (JP); Masaaki HAYAMA, Nara (JP); Akina TANEMURA, Kyoto (JP)

(73) Assignee: PANASONIC CORPORATION, Osaka (JP)

(21) Appl. No.: 14/019,195

(22) Filed: Sep. 5, 2013

Related U.S. Application Data
(63) Continuation of application No. PCT/JP2012/002383, filed on Apr. 5, 2012.

(30) Foreign Application Priority Data
Apr. 8, 2011 (JP) 2011-086040
Jul. 6, 2011 (JP) 2011-149820

(51) Int. Cl. G01N 33/50

(52) U.S. Cl. CPC G01N 33/5091
USPC 435/6.15; 435/287.2

(57) ABSTRACT

A diagnosis kit is configured to detect whether or not an extraneous organism exists in a red blood cell by using a biological specimen containing the red blood cell, and a stain solution capable of staining nucleic acid. The kit includes at least one diagnosis plate. The diagnosis plate includes a first chamber configured to store the stain solution and to have the biological specimen injected into the stain solution, a channel connected to the first chamber, and a test plate connected to the channel. A second chamber is connected with the test plate. The channel is configured to extract the red blood cell. The second chamber can collect a part of the biological specimen and a part of the stain solution. This diagnosis kit can detect extraneous organisms in the red blood cells easily with a small amount of the biological specimen.

This application is a continuation of International Application PCT/JP2012/002383, filed on Apr. 5, 2012, claiming the foreign priority of Japanese Patent Application No. 2011-086040, filed on Apr. 8, 2011, and Japanese Patent Application No. 2011-149820, filed on Jul. 6, 2011, the contents of which are incorporated therein by reference.

### TECHNICAL FIELD

The present invention relates to a diagnosis kit and a method of using the diagnosis kit used for an apparatus configured to extract or separate blood corpuscular components contained in whole blood of human and animal, or other tissue-derived fluids, and to perform diagnosis, for example, an apparatuses configured to extract red blood cells and diagnose a disease with which the blood cells are infected.

### BACKGROUND ART

Separating only a specific substance from a sampled specimen containing plural kinds of substances is necessary in various fields. However, it is difficult to detect only a specific substance among a lot of substances contained in a specimen especially when a content of the contained substance is small.

A case hardly detecting a specified substance of small content ratio will be described below. There are such diseases as malaria and babesia that are caused by protozoa parasitic on red blood cells, for instance, as the diseases related to the red blood cells, and there are methods of observing the red blood cells extracted from blood in order to diagnose these diseases accurately and at early stages. After infecting a human via anopheles, the malaria, or plasmodium become parasitic on the red blood cells and increase rapidly in population, and cause high fever, headache, nausea and the like illness. In conventional methods for detecting chemical compounds, such as antibodies, produced in the blood as a result of the infection, it is difficult to make diagnosis at an early stage since the chemical compounds do not have a detectable density until the population increases to a considerable size. For this reason, the red blood cells carrying the parasitic protozoa are observed directly and the number counted to detect a condition of the infection at the earliest possible stage. The accuracy of this method is extremely high since both uninfected red blood cells and infected red blood cells are counted.

The red blood cells can be observed by the following method. A blood specimen is prepared by applying a collected and controlled whole blood sample to a slide glass, and drying the sample blood. Then, the specimen is Giemsa-stained and observed by microscopic examination for the presence of red blood cells that are infected with malaria. In this method, nucleic acid of the malaria protozoa is stained by the Giemsa staining.

On the other hand, red blood cells generally have no nucleic acid, and it is therefore possible to observe and determine whether or not the red blood cells are infected with malaria protozoa by observing a degree of staining of the red blood cells. Since this method can be carried out only by observing the stain, it is easily determinable as to whether or not the red blood cells are parasitized with the malaria protozoa.

However, there needs a high degree of technique to manage both the early-stage diagnosis and a high detectability since the content of the red blood cells parasitized with the malaria protozoa is 0.05% or less to the entire population in the early stage. A factor that hampers improvement in the detectability of the blood cells in the method of direct observation will be described below. Since white blood cells, i.e., one kind of the blood cells exist in the whole blood have nucleic acid, it is necessary to make distinction of the Giemsa-stained blood cells out of the white blood cells contained originally in the whole blood and the red blood cells parasitized with the malaria protozoa. This work requires a high level of technique or an expensive apparatus. In addition, the micrographic observation that stained nucleic acid derived from the white blood cells may be misjudged as the nucleic acid derived from the malaria protozoa, which results in poor accuracy. Furthermore, it causes a substantial increase in the examination cost when counting is made by highly-skilled examination personnel in the cause of micrographic observation, besides such factors that the examination speed and the accuracy depend largely upon competency of the examination personnel.

Another method is available to cut the examination cost, in which a centrifugation method is used in advance to extract only red blood cells from the sample of whole blood and remove other components, such as white blood cells, that impede detection of the nucleic acid derived from the malaria protozoa, and ten, the biological specimen is examined with a detector.

In the above method of detection, optical measurement is performed by using a fluorescence reagent. In this measurement, erroneous judgment may be made due to differences in density of the biological specimen if spread excessively over a measuring position, which decreases the accuracy.

Particularly when detecting the malaria protozoa, it is required to measuring accuracy that the biological specimen is spread substantially uniformly into a single layer over the measuring position on a test plate. For this purpose, excessive cells may be removed by pouring cleaning fluids, such as a buffer, culture medium, surface-active agent, or enzymes, with the test plate held tilted slightly.

A method similar to the above method is disclosed in Patent Literature 1.

The method of extracting red blood cells by the centrifugation requires a large amount of whole blood for the examination, especially for use in extracting the red blood cells, although it can provide an accurate measurement. This method is therefore not suitable for such applications as a simplified test that uses a little amount, e.g. about 1 microliter taken from a finger for the measurement.

Moreover, regions where the examinations for malaria and the like may be necessary, that sufficient supply of electric power is not available, and the infrastructure for maintaining and controlling expensive detecting apparatuses is not established adequately.

### CITATION LIST

### Patent Literature

Patent Literature 1: WO2010/027003

### SUMMARY OF THE INVENTION

A diagnosis kit is configured to detect whether or not an extraneous organism exists in a red blood cell by using a biological specimen containing the red blood cell, and a stain solution capable of staining nucleic acid. The kit includes at least one diagnosis plate. The diagnosis plate includes a first chamber configured to store the stain solution and to have the biological specimen injected into the stain solution, a channel connected to the first chamber, and a test plate connected to the channel. A second chamber is connected with the test plate. The channel is configured to extract the red blood cell. The second chamber can collect a part of the biological specimen and a part of the stain solution.

This diagnosis kit can detect extraneous organisms in the red blood cells easily with a small amount of the biological specimen.

### BRIEF DESCRIPTION OF DRAWINGS

FIG. 1 is a top view of a diagnosis kit according to Exemplary Embodiment 1.

FIG. 2 is a sectional view of the diagnosis kit at line 2-2 shown in FIG. 1.

FIG. 3A is an enlarged view of a main part of the diagnosis kit according to Embodiment 1.

FIG. 3B is an enlarged view of a main part of the diagnosis kit according to Embodiment 1.

FIG. 4A is a sectional view of a wall of the diagnosis kit according to Embodiment 1.

FIG. 4B is a sectional view of another wall of the diagnosis kit according to Embodiment 1.

FIG. 4C is a sectional view of still another wall of the diagnosis kit according to Embodiment 1.

FIG. 4D is a sectional view of a further wall of the diagnosis kit according to Embodiment 1.

FIG. 4E is a sectional view of a further wall of the diagnosis kit according to Embodiment 1.

FIG. 5 is a top view of the diagnosis kit according to Embodiment 1.

FIG. 6 is a sectional view of the diagnosis kit according to Embodiment 1 for illustrating a method of using the diagnosis kit.

FIG. 7 is a schematic view of the diagnosis kit according to Embodiment 1 for illustrating the method of using the diagnosis kit.

FIG. 8 is a sectional view of the diagnosis kit according to Embodiment 1 for illustrating a method of manufacturing the diagnosis kit.

FIG. 9 is a sectional view of a diagnosis kit according to Exemplary Embodiment 2.

FIG. 10 is a top view of a main part of the diagnosis kit according to Embodiment 2.

FIG. 11 is a top view of a cavity of the diagnosis kit according to Embodiment 2.

FIG. 12 is a sectional view of the cavity at line 12-12 shown in FIG. 11.

FIG. 13 is a sectional view of another cavity of the diagnosis kit according to Embodiment 2.

FIG. 14A is a top view of another test plate of the diagnosis kit according to Embodiment 2.

FIG. 14B is a top view of still another test plate of the diagnosis kit according to Embodiment 2.

FIG. 15 is a sectional view of a diagnosis plate of a diagnosis kit according to Exemplary Embodiment 3.

FIG. 16 is a sectional view of a diagnosis plate of a diagnosis kit according to Exemplary Embodiment 4.

FIG. 17 is a schematic view of a detecting apparatus according to Exemplary Embodiment 5.

FIG. 18 is a top view of a test plate of a diagnosis kit according to Exemplary Embodiment 6.

FIG. 19 is an enlarged view of a main part of the test plate according to Embodiment 6.

FIG. 20 is a sectional view of the test plate at line 20-20 shown in FIG. 19.

### DETAIL DESCRIPTION OF PREFERRED EMBODIMENTS

### Exemplary Embodiment 1

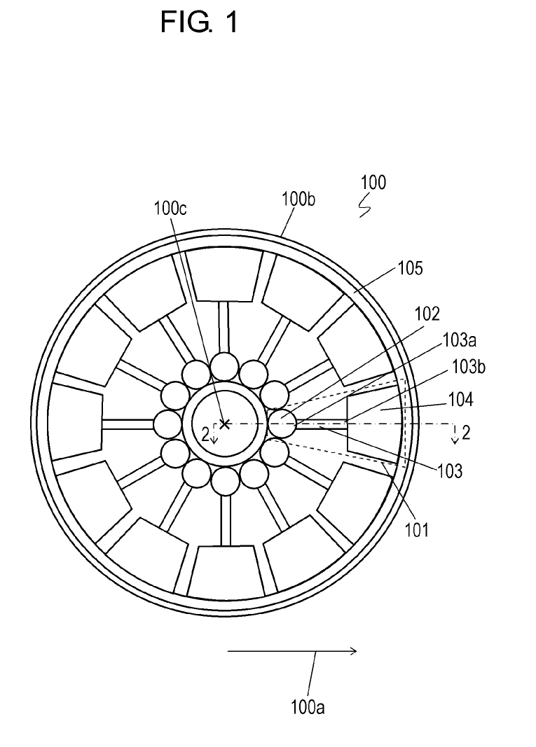

FIG. 1 is a top view of diagnosis kit 100 according to Exemplary Embodiment 1. Diagnosis kit 100 includes base plate 100b and diagnosis plates 101 disposed to base plate 100b. Base plate 100b has a circular shape about center axis 100c. Diagnosis plates 101 are arranged radially at equiangular intervals about center axis 100c to extend in predetermined directions 100a away from center axis 100c. Diagnosis plates 101 enable diagnosis kit 100 to measure (examine) plural biological specimens at once.

Diagnosis plates 101 can execute a process of diluting a biological specimen and staining nucleic acid thereof, a process of extracting red blood cells, i.e., separating the red blood cells from white blood cells and extract the red blood cells, and a process of disposing the extracted red blood cells. Diagnosis kit 100 can extract a target substance, the red blood cells, from the biological specimen containing the target substance, and examining the target substance.

The examination of the red blood cells include detecting whether or not extraneous organisms exist in the red blood cells, that is, determining whether or not red blood cells infected with pathogenic microorganisms exist, by using a biological specimen containing the red blood cells. For instance, diagnosis kit 100 can diagnosis whether or not malaria protozoa has intruded into red blood cells, parasitized and infected the red blood cells with the malaria.

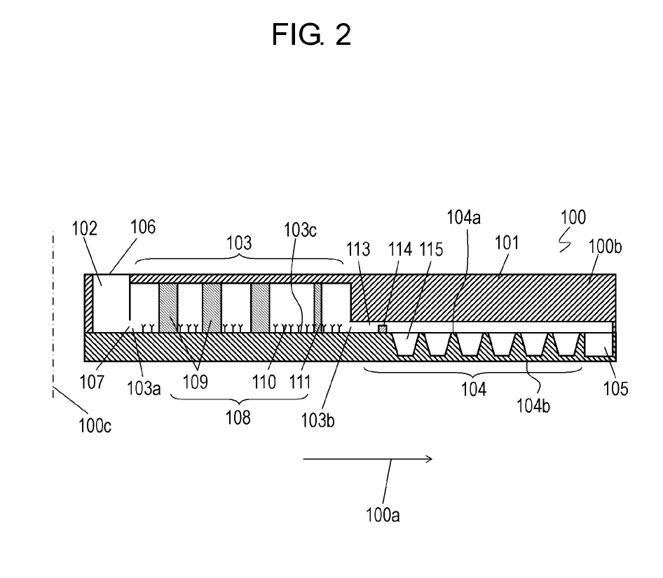

FIG. 2 is a sectional view of diagnosis kit 100 at line 2-2 shown in FIG. 1. Each of diagnosis plates 101 includes chamber 102, channel 103, and test plate 104 which are arranged in this order from one end closer to center axis 100c. Chamber 102 is configured to dilute the biological specimen, and stain the nucleic acid of the specimen. Chamber 102 has through-hole 107 formed in a wall thereof. Channel 103 has one end 103a connected with chamber 102 via through-hole 107, and another end 103b opposite to end 103a, and is configured to extract red blood cells from the biological specimen. Test plate 104 is connected to end 103b of channel 103, and configured to having the extracted red blood cells placed thereon. Plural diagnosis plates 101 are independent from one another. Chamber 105 is provided at an outer periphery of an annular shape of base plate 100b, and is connected with outer ends of test plates 104. Chamber 105 is connected with all the plural diagnosis plates 101. Channel 103 extends in predetermined direction 100a from one end 103a to another end 103b.

Test plate 104 has surface 104a and surface 104b opposite to surface 104a. Plural cavities 115 are formed in surface 104a.

Base plate 100b has a circular ring shape according to Embodiment 1, but may have another shape, such as a polygonal ring shape. Diagnosis kit 100 having such a shape can be fixed to a carrier table.

Single diagnosis kit 100 according to Embodiment 1 includes plural diagnosis plates 101, as shown in FIG. 1. Alternatively, single diagnosis kit 100 may have at least one diagnosis kit 100. However, single diagnosis kit 100 including plural diagnosis plates 101 can examine plural biological specimens at once, thereby diagnosing more efficiently for a shorter time.

A stain solution for staining the target substance and a diluent for diluting the biological specimen can be previously stored in chamber 102. The biological specimen containing red blood cells, i.e., the target substance, is injected into chamber 102 storing the stain solution and the diluent therein to stain specific cells and dilute the biological specimen.

In the case that the stain solution and the diluent are previously stored in chamber 102, base plate 100b may preferably have film 106 to cover at least an upper side of chamber 102. In this case, the biological specimen is injected into chamber 102 by piercing film 106 with, e.g. a pipet or a syringe.

To detect blood cells infected with pathogenic microorganisms out of the biological specimen containing red blood cells, nucleic acid of the pathogenic microorganisms in the infected blood cells is previously fluorescence-stained. In the biological specimen containing infected red blood cells, for instance, the infected red blood cells are marked by staining the nucleic acid of the infecting microorganisms, such as protozoa, with fluorescence stain. Additionally, the red blood cells may be marked with, e.g. a fluorescent substance if necessary.

The target substance can be stained more efficiently by circulating the liquid solution inside chamber 102 during this fluorescence staining. The biological specimen containing red blood cells may have a high viscosity if it is blood. In this cases, a diluent may be added into chamber 102 together with the stain solution to decrease the viscosity of the biological specimen inside of chamber 102, so that the red blood cells can be extracted easily thereafter.

Besides, the biological specimen may be preferably mixed with the stain solution and the diluent in chamber 102 by applying vibration.

The biological specimen, the stain solution and the diluent can be mixed by making a pipetting motion several times to circulate the biological specimen inside chamber 102 when injecting the biological specimen into chamber 102.

The biological specimen, the stain solution and the diluent can be mixed more efficiently by applying the vibration mentioned above after the pipetting motion.

Alternatively, a stirring bar for mixing the biological specimen, the stain solution and the diluent may be previously enclosed inside chamber 102. The biological specimen, the stain solution and the diluent can be mixed more efficiently by moving the stirring bar to stir the biological specimen, the stain solution and the diluent.

The biological specimen may be preferably incubated for several minutes after suspending the liquid solution inside chamber 102.

The Giemsa's staining, acridine-orange staining, Wright's staining, Jenner's staining, Leishman's staining and Romanowsky's staining are some examples of staining methods available for use, and suitable stain solutions can be used according to the individual staining methods. Alternatively, a stain identified by SYTO59® can be used as a stain solution for malaria-infected red blood cells.

The Giemsa's staining is used to examine infection with malaria protozoa. When the Giemsa's staining is used, the stain solution can be prepared by just adding 1 drop to 1½ drops of undiluted Giemsa's stain solution into 1 mL of 10-mM phosphate buffer (pH 7.2 to 7.4), and by mixing them. An acidulous buffer solution of about pH 6.4 is often used to observe a blood profile. The stain solution adjusted to have pH 7.2 to 7.4 is rather suitable to observe the shape of malaria protozoa. This stain solution changes the color of infected red blood cells into bluish color.

In the acridine-orange staining, for example, the stain solution can be prepared by dissolving 5.0 mg of acrylic orange and 2.5 g of glycerin into 47.5 mL of 10-mM phosphate buffer (pH 7.2 to 7.4). This stain solution causes nuclei of the infected red blood cells to generate yellow fluorescence when excited by excitation light having a wavelength ranging from 450 nm to 490 nm.

In the case that the type SYTO59® is used as a stain solution, for example, nucleic acid of the malaria protozoa in the infected red blood cells is fluorescence-stained, and causes the nucleic acid to generate fluorescence. This stain solution causes the fluorescence of a wave length ranging from 640 nm to 660 nm to be generated when excited with excitation light having a wavelength from 600 nm to 635 nm.

The diluent may be a substance, such as a buffering solution, isotonic solution, culture medium, or surface-active agent, that does not denature the cells contained in the biological specimen.

Alternatively, an anticoagulant, such as ethylene-diamine-teraacetic acid (EDTA), may be used. However, any of heparin-group anticoagulants is not suitable since they adversely influence the staining when the Giemsa's stain is applied.

The biological specimen marked by the above method is stored in chamber 102 together with a liquid solution containing the stain solution and the diluent previously enclosed in chamber 102, as a sample liquid.

Next, the sample liquid is introduced into channel 103 connected with chamber 102. During this step, diagnosis kit 100 is rotated about center axis 100c so that the red blood cells contained in the sample liquid can be moved efficiently from chamber 102 to test plate 104 through channel 103 by a centrifugal force.

In the diagnosis kit according to Embodiment 1, the red blood cells are moved from chamber 102 to test plate 104 by the centrifugal force, this is not restrictive. The red blood cells may be moved from chamber 102 to test plate 104 by producing a difference in, e.g. pressure, between chamber 102 and channel 103 instead.

The difference in the pressure can be produced between chamber 102 and channel 103 by applying a pressure to chamber 102. Chamber 102 is pressurized from above as one method of applying the pressure to chamber 102.

The difference in the pressure can be produced between chamber 102 and channel 103 by decompressing from test plate 104 and chamber 105. A method of decompressing the pressure of channel 103 may be to discharge air by suctioning air test plate 104 and chamber 105.

Chamber 102 has through-hole 107 therein to connect chamber 102 with channel 103. Through-hole 107 is formed preferably in the lower part of the wall between chamber 102 and channel 103 from view of the productivity. A part of through-hole 107 has a hydrophobic treatment. The liquid enclosed inside chamber 102 is retained in chamber 102 due to water repellency of the hydrophobic treatment when diagnosis kit 100 stops. The sample liquid can be introduced into channel 103 via through-hole 107 only when diagnosis kit 100 is rotated. Specifically, the liquid can be retained inside chamber 102 without flowing into through-hole 107 due to the surface tension if the sectional area of through-hole 107 is small enough as not to be greater than one-half of the sectional area of channel 103 and a part of through-hole 107 has the hydrophobic property.

The part of through-hole 107 may be made of a hydrophobic material or processed with a hydrophobic treatment to have the water-repellent property.

The hydrophobic treatment may be applied entirely to a surface of through-hole 107 although the hydrophobic treatment is preferably provided only at a part of an opening of through-hole 107 communicating with chamber 102. The liquid inside chamber 102 can be retained securely at not only one end of through-hole 107 but at the entire through-hole 107 in the case that the entire inner wall surface of through-hole 107 has the hydrophobic property.

The sample liquid inside chamber 102 can be delivered to channel 103 via through-hole 107 by rotating diagnosis kit 100 for a predetermined time if the entire inner wall surface of through-hole 107 has the hydrophobic property. That is, the sample liquid can be delivered reliably to channel 103 by controlling a rotation speed and a rotation time.

Alternatively, the entire inner walls of chamber 102 and channel 103 including through-hole 107 may have a hydrophobic property. The entire inner wall surfaces in an area around channel 103 can have the hydrophobic property easily by using base materials made of a hydrophobic material to fabricate diagnosis kit 100 by bonding two base materials together. The productivity of diagnosis kit 100 can thus be improved.

The hydrophobic material may be a semiconductor material, such as single-crystal silicon, amorphous silicon, silicon carbide, silicon oxide or silicon nitride, an inorganic insulation material, such as alumina, sapphire, forsterite, silicon carbide, silicon oxide or silicon nitride, and an organic material such as polyethylene, ethylene, polypropylene, polyisobutylene, polyethylene terephthalate (PET), unsaturated polyester, fluorine-containing resin, polyvinyl chloride, polyvinylidene chloride, polyvinyl acetate, polyvinyl alcohol, polyvinyl acetal, acrylic resin, polyacrylonitrile, polystyrene, acetal resin, polycarbonate (PC), polyamide, phenolic resin, urea resin, epoxy resin, melamine resin, styrene acrylonitrile copolymer, acrylonitrile butadiene styrene copolymer, silicone resin, polyphenylene oxide, or polysulfone. The PET and PC may be suitably used as the hydrophobic materials.

The process of hydrophobic treatment may be coatings of fluoro-resin and coating of silicone resin. The fluoro-resin coating is suitably used.

A contact angle of the hydrophobic portion of through-hole 107 with still water is preferably not smaller than about 35° and not larger than 120°. The sample liquid can infiltrate by itself into channel 103 from chamber 102 due to a capillary action even when diagnosis kit 100 is not rotated, if the wall of channel 103 has a contact angle not larger than about 35°. On the other hand, the biological specimen may not infiltrate into channel 103 from chamber 102 even when the centrifugal force is exerted upon diagnosis kit 100 with a highest rotation speed since the water repellency of the hydrophobic portion of through-hole 107 becomes excessively high if the hydrophobic portion has a contact angle not smaller than about 120°.

The hydrophobic portion of a part of through-hole 107 has the contact angle with still water ranging from 66° to 90°.

Wall surfaces (i.e., side and bottom surfaces) of chamber 102 other than the hydrophobic portion of through-hole 107 may have either hydrophobicity or hydrophilicity. The wall surfaces having the hydrophilicity can facilitate infiltration of the liquid flowing from chamber 102 to channel 103 more reliably due to the wetting effect and capillary action.

The hydrophilicity can be provided by allowing the portion to be made of a hydrophilic material or by applying a hydrophilic treatment to the portion.

The hydrophilic material may be glass, silica glass, or metallic materials, such as aluminum, copper, or stainless steel. However, surfaces of metallic material are cleaned before being used by removing organic substances adhered to the surfaces. The hydrophilic treatment may be a coating of surface-active agent typified by Triton X, and a high molecular compound having hydrophilic group such as hydroxyl group, sulfonic group and carboxyl group. Coating of a surface-active agent is rather preferable. However, the above hydrophilic material is treated not to hydrophilize the inner wall of through-hole 107. In particular, the hydrophilic material may be eluted from the walls of chamber 102, and may hydrophilize the inner wall of through-hole 107. A hydrophilic material that can be less removable from the adhered surface is preferably used for this reason.

Target-substance path 108 is provided on at least a part of the inner wall of channel 103.

Channel 103 may preferably have a width ranging from about 10 μm to 1 mm so that the red blood cells can be extracted quickly.

Target-substance path 108 includes non-target substance trap 109 for capturing white blood cells. Non-target substance trap 109 is made of fibrous materials 109a.

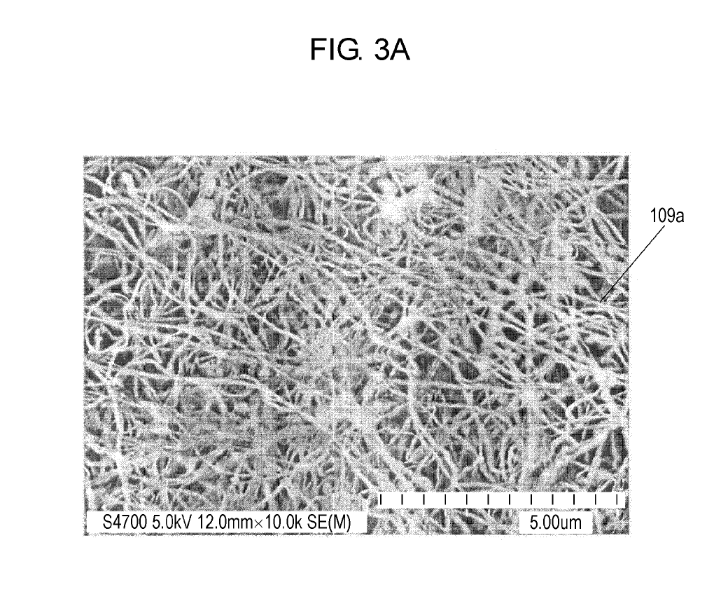

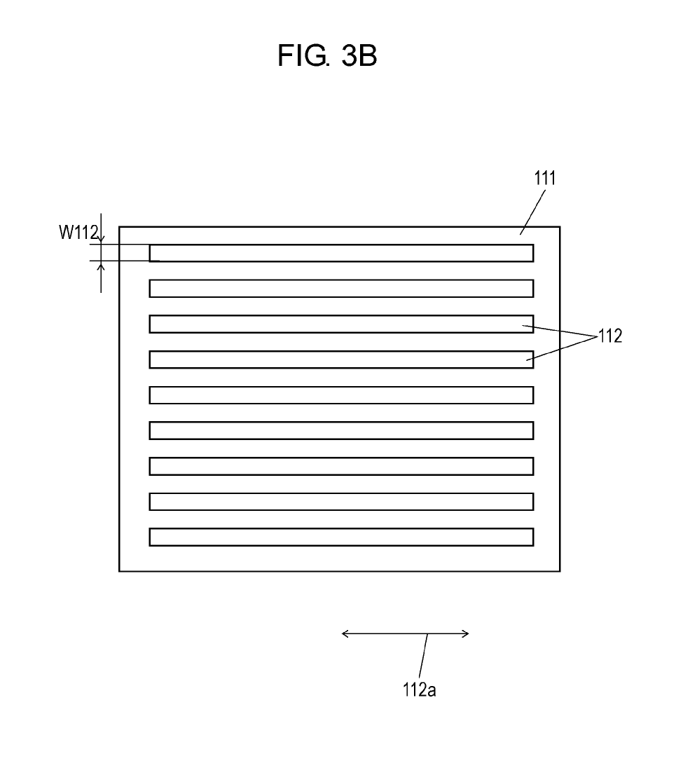

FIG. 3A is an enlarged view of a main part of diagnosis kit 100, an SEM photograph of fibrous materials 109a. Fibrous materials 109a are made of oxidized silicon mainly containing, .e.g, silicon oxide, and are made preferably of silicon dioxide of an amorphous form. Fibrous materials 109a have thicknesses ranging from about 0.01 μm to 1 μm. One ends of fibrous materials 109a are directly joined to an inner wall of channel 103. Fibrous materials 109a densely exist to be tangled with one another. Fibrous materials 109a include fibers branched irregularly to different directions. Here, the expression “directly joined” means that fibrous materials 109a are formed directly on the inner wall of channel 103 such that the atoms or the molecules composing the inner wall of channel 103 and fibrous materials 109a are directly bonded, and it commonly indicates the state of covalent bonding between the molecules. In the case that a surface of the inner wall of channel 103 is made of silicon, fibrous materials 109a are formed on the inner wall surface of channel 103 by using the silicon as a raw material so that the silicon atoms in the inner wall surface of channel 103 and the silicon atoms in fibrous materials 109a can become directly joined by establishing covalent bonding via oxygen molecules in the atmosphere for fabricating fibrous materials 109a.

Fibrous materials 109a can be constructed firmly on the inner wall of channel 103 since they are tangled with one another, and branched into twigs. In addition, a lot of spaces formed by fibrous materials 109a can be easily filled from various directions since fibrous materials 109a are individually curved and entangled with one another.

Fibrous materials 109a preferably have different thicknesses rather than a uniform thickness.

In this case, fibrous materials 109a having smaller diameters fill spaces provided between fibrous materials 109a having larger diameters, thereby providing fibrous materials 109a with fine spaces as a whole. This structure becomes phenomenal especially when the number of fibrous materials 109a is kept unchanged.

The shortest distance of each space between adjoining fibrous materials 109a is smaller than the diameter of trapped substances, such as white blood cells.

Non-target substance trap 109 is made of plural fibrous materials 109a mutually entangled. Among solutes contained in the biological specimen, the white blood cells and other substances having maximum diameter larger than the spaces among fibrous materials 109a are captured by fibrous materials 109a as the substances to be trapped. On the other hand, the red blood cells capable of passing through the spaces among fibrous materials 109a pass through fibrous materials 109a, thereby enabling non-target substance trap 109 to extract the red blood cells. Since the red blood cells have deformability that makes them easily deformable, the red blood cells can pass through these spaces even if the spaces among fibrous materials 109a are narrower than sizes of the red blood cells, thus being extracted.

To extract the red blood cells, non-target substance trap 109 may preferably to has spaces ranging from 3 μm to 6 μm among fibrous materials 109a. Diameters of the red blood cells range from 7 μm to 8 μm, and the red blood cells can enter and pass through capillary vessels of diameters as small as one-half their own diameters or even less. Diameters of the white blood cells range from 6 μm to 30 μm, and the white blood cells have smaller deformability than the red blood cells. Fibrous materials 109a of non-target substance trap 109 that form the spaces ranging from 3 μm to 6 μm allow only the red blood cells to pass through while not allowing the white blood cells to pass through.

Fibrous materials 109a may be preferably applied and coated previously with a reagent that destroys only the white blood cells so that the white blood cells can be separated more efficiently. Alternatively, fibrous materials 109a may preferably be applied and coated previously with antibody that adsorbs only certain proteins exposed only on surfaces of the white blood cells, so that the white blood cells can be separated even more effectively. Fibrous materials 109a may be treated with an organic solvent or subjected to drying under high temperatures in association with the coating of such a reagent. Fibrous materials 109a made of silicon dioxide have a resistance to chemicals so that fibrous materials 109a can be coated with reagents of various properties. In this case, fibrous materials 109a have a high heat resistance, and hence, can be coated with such reagents without melting and damaging the shape of fibrous materials 109a during treatment with high temperatures. As discussed, fibrous materials 109a made of silicon dioxide can easily have coating and fixing of the adsorbent since they have a superior chemical resistance and heat resistance to fibrous materials 109a made of organic polymer.

Non-target substance trap 109 may be made of a porous material. The porous material may be nitrocellulose, polyvinylidene fluoride (PVDF) and agarose. The porous material may be made by forming a lot of through-holes in an inorganic substrate, such as silicon, glass, or ceramic.

Non-target substance trap 109 made of a porous material preferably has pore diameters ranging from about 3 μm to 6 μm. Non-target substance trap 109 having the pore diameters ranging from 3 μm to 6 μm allows only the red blood cells to pass through non-target substance trap 109 without allowing the white blood cells to pass through non-target substance trap 109 for the same reason as discussed above.

Alternatively, non-target substance trap 109 may have both fibrous materials 109a and the porous material mentioned above.

Alternatively, target-substance path 108 may have non-target substance capturing probes 110 fixed onto the inner wall of channel 103. Non-target substance capturing probes 110 are made of anti-leukocyte antibody, such as anti-HLA antibody, anti-granulocyte antibody, or monoclonal antibody designated as CD8 and CD4, and capable of capturing only the white blood cells.

Various coupling reactions, such as silane coupling reaction, are available to fix non-target substance capturing probes 110. Non-target substance capturing probes 110 can be fixed by causing a silane coupling agent having an acid anhydride functional group, such as 3-(tri-ethoxysilyl) propyl succinic anhydride, to contact a solid phase support, and then, by a coupling process of bioactive substance to the acid anhydride functional group while maintaining the solid phase support within a temperature range from 0° C. to 60° C.

In the case that the inner wall of channel 103 is made of a metal, such as gold or platinum, a self-assembled monomolecular (SAM) can be used.

The above method of using chemical reaction is not only the method of fixing non-target substance capturing probes 110, but it is also possible to fix non-target substance capturing probes 110 to channel 103 by a process not associated with any chemical reaction. For example, non-target substance capturing probes 110 and channel 103 can be joined directly by making plasma treatment of channel 103.

Non-target substance capturing probes 110 are fixed to the inner wall of channel 103, but may be fixed beforehand to beads made of, e.g. polystyrene. The beads are then secured onto the inner wall of channel 103. In this instance, a lot of beads have non-target substance capturing probes 110 fixed inside channel 103.

The beads can be fixed to the inner wall of channel 103 by applying UV-radiation after the beads fixed to non-target substance capturing probes 110 and a UV curing resin are mixed and disposed to the inner wall of channel 103.

Target-substance path 108 may include permeable plate 111 extending in a direction perpendicular to a flow direction in which the sample solution flows in channel 103.

FIG. 3B is an enlarged view of a main part of diagnosis kit 100, and particularly illustrates permeable plate 111. Permeable plate 111 has plural slits 112 therein elongating in longitudinal direction 112a. Slits 112 preferably have width W112 not larger than 7 μm in a direction perpendicular to longitudinal direction 112a. Permeable plate 111 allows the red blood cells to pass more efficiently by having width W112 not larger than 7 μm. The red blood cells can pass through slits 112 sue to deformability of the red blood cells even if width W112 is smaller than diameters of the red blood cells, thereby extracting the red blood cells selectively. However, width W112 of slits 112 is larger than a size through which the red blood cells are unable to pass even when the red blood cells deform. Width W112 is preferably not smaller than 3 μm in consideration of the extracting efficiency.

Permeable plate 111 has slits 112, as shown in FIG. 3B, but may have at least one slit 112 formed therein for the purpose. However, the red blood cells can pass more efficiently by forming plural slits 112.

Target-substance path 108 includes non-target substance traps 109 and non-target substance capturing probes 110 formed on the inner walls of channel 103. Non-target substance traps 109 and non-target substance capturing probes 110 may be preferably arranged alternately along channel 103. This structure can trap white blood cells from the sample liquid more efficiently by disposing target-substance path 108 at plural locations on the inner walls of channel 103.

According to Embodiment 1, target-substance path 108 includes non-target substance traps 109, non-target substance capturing probes 110, and permeable plate 111, all disposed to the inner walls of channel 103. Target-substance path 108 can provide similar effects if target-substance path 108 has only one or a combination of any two of non-target substance trap 109, non-target substance capturing probe 110, and permeable plate 111.

The width of channel 103 becomes narrower gradually as increasing of the distance from chamber 102. This structure can extract the red blood cells more efficiently in channel 103.

The height of channel 103 decreases gradually as the a distance from chamber 102 increase. This structure allows channel 103 to extract the red blood cells more efficiently in channel 103. A height of channel 103 near one end 103b thereof or a height of channel 103 at a portion where channel 103 is connected to test plate 104 is more preferably not larger than 7 μm. This structure can efficiently pass the red blood cells to test plate 104.

The red blood cells thus extracted are moved to test plate 104 connected with another end 103b of channel 103. To be concrete, a red-blood-cell adjusted liquid can be obtained from the sample liquid stored in chamber 102 having the sample liquid in which the white blood cells are removed while passing through channel 103.

Diagnosis plate 101 may preferably have a liquid blocking structure for collecting the liquid from channel 103.

Diagnosis plate 101 may have liquid reservoir 113 functioning as the liquid blocking structure. Liquid reservoir 113 is constituted by bottom 103c of channel 103 and barrier 114 provided near an outlet of channel 103 or at a joint portion where channel 103 is connected with test plate 104. The red-blood-cell adjusted liquid is temporarily collected in liquid reservoir 113. When a predetermined amount of centrifugal force or a pressure is applied to the collected red blood cells, the red-blood-cell adjusted liquid is forced to flow over barrier 114, so that the red blood cells can be distributed efficiently to test plate 104. Barrier 114 may has a cross section having a rectangular shape along direction 100a, as shown in FIG. 2.

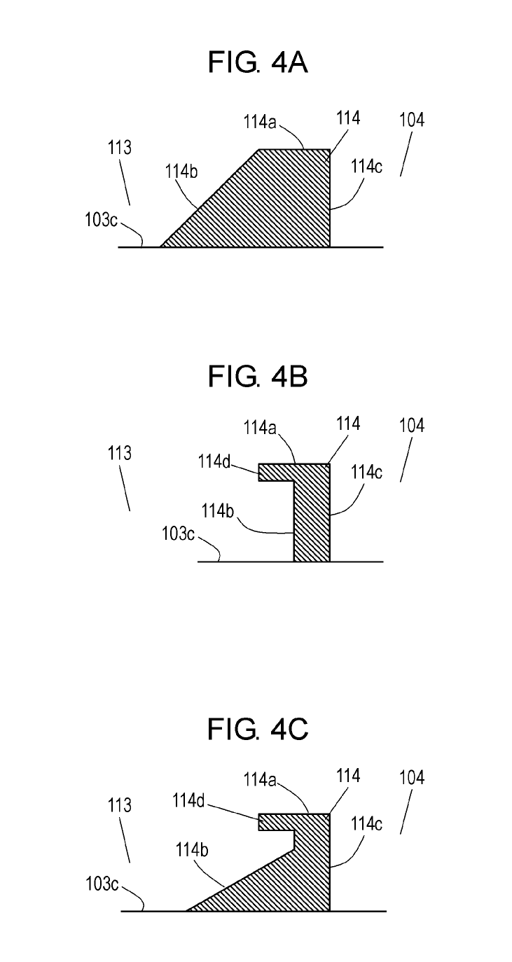

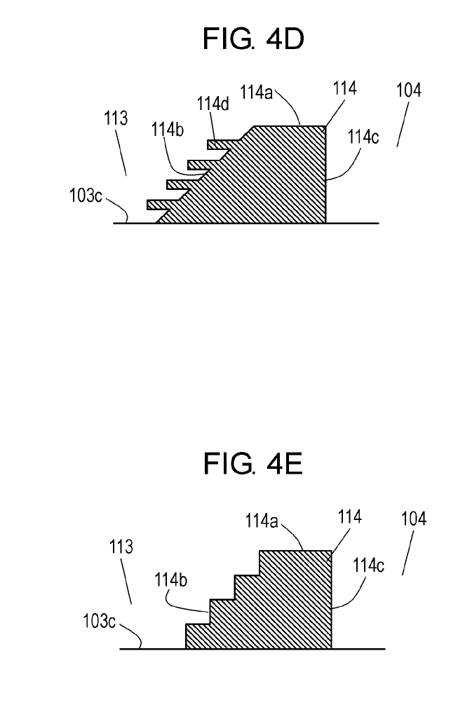

FIG. 4A to 4E are sectional views of barriers 114, and illustrate cross sections of barriers 114 along direction 100a. Each of barriers 114 has side surface 114b facing liquid reservoir 113, side surface 114c facing test plate 104, and upper surface 114a. In barrier 114 shown in FIG. 4A, side surface 114b facing liquid reservoir 113 preferably inclines toward bottom 103c, and side surface 114c is preferably perpendicular to bottom 103c, so that barrier 114 has a cross section tapering toward upper surface 114a from bottom 103c. The red-blood-cell adjusted liquid collected in liquid reservoir 113 can be poured efficiently into test plate 104 due to this shape.

Side surface 114b of barrier 114 preferably has water repellency while upper surface 114a preferably has hydrophilic property. The red-blood-cell adjusted liquid flown into liquid reservoir 113 forms a liquid drop due to the surface tension on side surface 114b. When the red-blood-cell adjusted liquid of even a small amount rises over barrier 114 upon having a centrifugal force or a pressure applied thereafter, the remaining portion of the red-blood-cell adjusted liquid also rises over barrier 114 by the capillary action, and flows toward test plate 104.

Barrier 114 shown in FIG. 4B has protruding portion 114d that protrudes from side surface 114b facing liquid reservoir 113. Side surface 114b is perpendicular to bottom 103c.

Barrier 114 shown in FIG. 4C has protruding portion 114d that protrudes from side surface 114b facing liquid reservoir 113. Side surface 114b inclines toward bottom 103c such that barrier 114 has a tapering cross section.

Barrier 114 shown in FIG. 4D has plural protruding portions 114d that protrude from side surface 114b facing liquid reservoir 113. Side surface 114b inclines toward bottom 103c such that barrier 114 has a tapering cross section.

In barrier 114 shown in FIG. 4E, side surface 114b has a stair shape having plural steps. In any of barriers 114 shown in FIG. 4A to 4E, the red-blood-cell adjusted liquid flown into liquid reservoir 113 can easily form a liquid drop due to the surface tension.

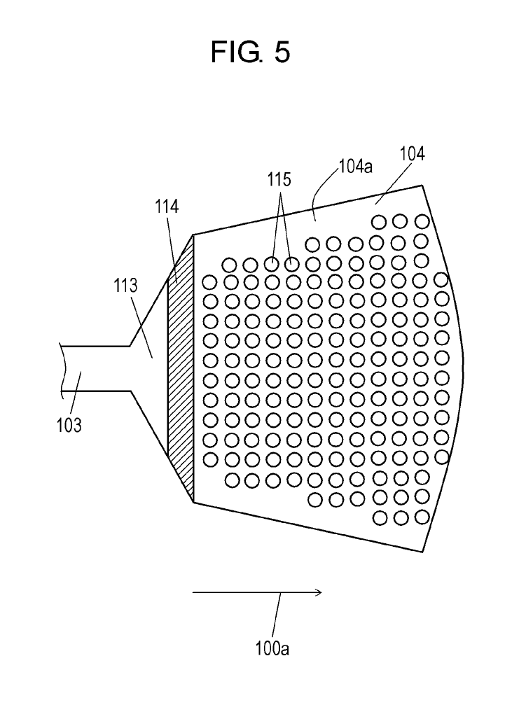

FIG. 5 is a top view of diagnosis kit 100, and particularly, illustrates channel 103 and test plate 104. Plural cavities 115 are formed in surface 104a of test plate 104. Red blood cells contained in the red-blood-cell adjusted liquid are placed in cavities 115. The red blood cells can be distributed efficiently into cavities 115 by keeping the red-blood-cell adjusted liquid collected temporarily within liquid reservoir 113 before being distributed to cavities 115 in test plate 104.

Test plate 104 can be implemented by a basal plate made of, for example, silicon, poly-silicon, glass, silicon oxide, SOI board, or a polymer, such as polyethylene, polystyrene, polypropylene, polyamide, polycarbonate, polydimethylsiloxane (PDMS), polymethyl methacrylate (PMMA), or cyclic olefin copolymer (COC), or a combination of materials, such as a laminate of glass and polymer.

Cavities 115 can be formed by one of etching process, photolithography, and electron beam lithography applied directly to the basal plate mentioned above, or by bonding film 106 having cavities 115 therein onto an upper surface of the basal plate.

Surface 104a and cavities 115 of test plate 104 may have a surface treatment performed thereto to have a hydrophilic property, when necessary, by, e.g. plasma treatment, oxygen plasma treatment, or corona discharge treatment. Hydrophilicity may be preferably provided by applying oxygen plasma or the like treatment when a hydrophobic substrate is used, for instance, as the basal plate.

The shape of Cavities 115 is not specifically limited, and may be a cylindrical shape, a semispherical shape, a polygonal pyramid shape, a rectangular parallelepiped shape, or a cubic shape.

According to Embodiment 1, each of cavities 115 has a size capable of accommodating hundred red blood cells therein. Test plate 104 may preferably have thousand or more, and even more preferably ten thousand cavities 115. In these cases, single test plate 104 can carry about hundred thousand red blood cells, and more preferably million red blood cells.

Diagnosis kit 100 is preferably rotated in this instance, to distribute the red blood cells evenly into cavities 115. During this rotation, it is more preferable to make such manipulation as changing the rotating speed and reversing the rotating direction. After the red blood cells are introduced into cavities 115, an unnecessary portion of the red-blood-cell adjusted liquid is moved to chamber 105 connected with test plates 104 connected with diagnosis plates 101, and is collected as waste liquid by continuing the rotation. Chamber 105 is preferably formed in the outermost periphery of diagnosis plates 101.

The red blood cells prepared in this manner are examined by fluorometric detection with a fluorescence microscope or micro-scanner, for example. It is detected whether or not infection and seriousness of the disease exists by examining intensity of fluorescence and the number of the marked red blood cells.

In the case that diagnosis kit 100 has a circular shape, plural cavities 115 in test plate 104 are preferably arranged in conformity with the outer periphery of base plate 100b. In other words, plural cavities 115 are preferably arranged along plural concentric circles about center axis 100c. The above configuration facilitates the examination of the red blood cells disposed into cavities 115 more efficiently in a short time.

A method of diagnosing the red blood cells by using diagnosis kit 100 and fluorescence will be described below.

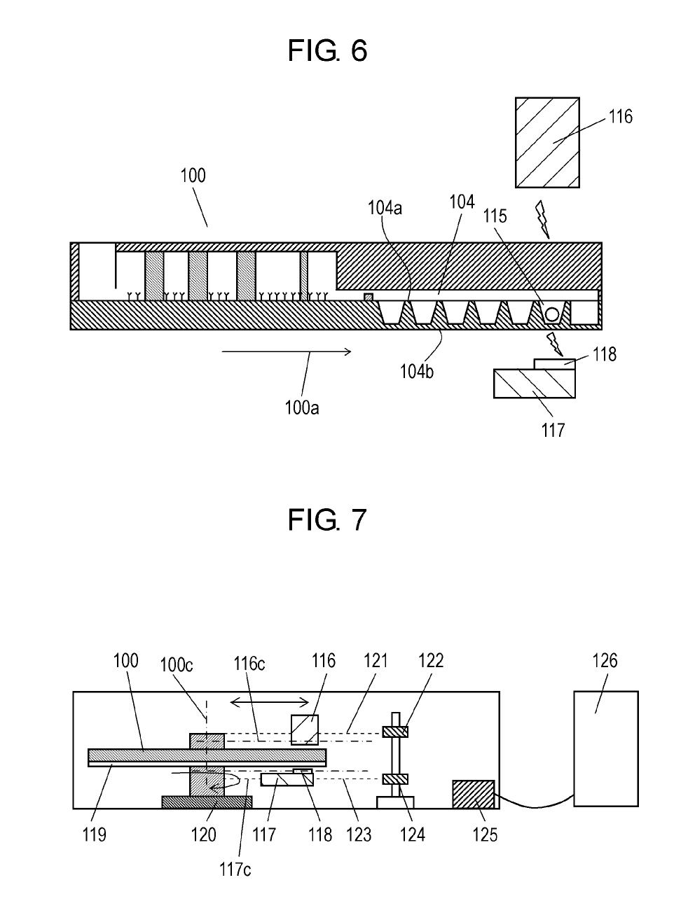

FIG. 6 is a sectional view of diagnosis kit 100 for illustrating a method of diagnosing the red blood cells by using the fluorescence. Laser module 116 is disposed above test plate 104 at a position facing surface 104a. Photo detector 117 is disposed below test plate 104 at a position facing surface 104b. Cavities 115 are irradiated with excitation light having a predetermined wavelength by laser module 116. The red blood cells in cavities 115 generate light due to the excitation light. The generated light is detected by photo detector 117.

Laser module 116 generates laser light having a wavelength suitable for the fluorescence reagent. When infected red blood cells exist among the red blood cells contained in cavities 115, fluorochrome from the infected red blood cells is excited by the radiated laser light, and produces fluorescence. An intensity of this fluorescence is detected by photo detector 117. Although photo detector 117 detects an intensity of the fluorescence according to Embodiment 1, it may diagnose the red blood cells by detecting whether or not fluorescence is produced.

In diagnosis kit 100 according to Embodiment 1, the red blood cells are irradiated with the light of excitation wavelength through one of surfaces 104a and 104b of test plate 104 to cause extraneous organisms in the red blood cells to produce fluorescence, and an intensity of the fluorescence from surface 104b is measured. In diagnosis kit 100, the intensity of the fluorescence can be measured at either one of surfaces 104a and 104b of test plate 104.

Additionally, fluorescence filter 118 may be preferably provided between diagnosis kit 100 and photo detector 117 to block the light having the excitation wavelength so as to avoid photo detector 117 from detecting unwanted laser light not exciting fluorescence. Fluorescence filter 118 is preferably provided at a light receiving section of photo detector 117. The unwanted laser light not exciting fluorescence can be prevented from being detected by photo detector 117 by measuring the intensity of the fluorescence through fluorescence filter 118, that is, by measuring the intensity of the fluorescence radiated from diagnosis kit 100 and passing through fluorescence filter 118.

In addition, a lens may be provided between diagnosis plate 101 and fluorescence filter 118 to raise the sensibility.

When a fluorescence reagent of SYTO59® is used, for instance, light is excited by visible light having a wavelength of 635 nm. In this case, photo detector 117 accurately detects light of wavelengths longer than 635 nm since the light generated by the reagent and detected has wavelengths longer than 635 nm when the visible light of 635 nm is radiated from laser module 116. In addition, fluorescence filter 118 can preferably block the light of wavelengths of about 635 nm, and pass the light of wavelengths not shorter than 645 nm without blocking it, thereby preventing photo detector 117 from failing to detect unwanted light that does not contribute to excitation of the fluorochrome.

A method of diagnosing red blood cells more efficiently with diagnosis kit 100 will be described below. FIG. 7 is a schematic view of diagnosis kit 100 for illustrating the method. Diagnosis kit 100 is fixed to an upper surface of rotatable carrier table 119. Carrier table 119 can be rotated by carrier table drive unit 120. A spindle motor, for instance, can be used as carrier table drive unit 120.

When a sample liquid is prepared in chamber 102 at first of diagnosis, a biological specimen, a stain solution and a diluent are mixed preferably by vibrating them in chamber 102, as previously described. After diagnosis kit 100 is fixed to carrier table 119, as shown in FIG. 7, diagnosis kit 100 can be rotated and vibrated by driving carrier table 119 with carrier table drive unit 120. This circulates the liquid solution in chamber 102 and mixes them effectively. At this moment, such manipulations as changing a rotating speed and reversing a direction of the rotation may be preferably executed.

A stirring bar may be preferably provide inside chamber 102 previously to mix the biological specimen, the stain solution and the diluent. The biological specimen, the stain solution and the diluent can be mixed more efficiently by moving the stirring bar to stir the biological specimen, the stain solution and the diluent. The stirring bar may be a magnetic stirrer. In this case, a magnet is preferably embedded in a location underneath chamber 102 of carrier table 119 when diagnosis kit 100 is fixed to carrier table 119.

In order to introduce the sample liquid more efficiently from chamber 102 to channel 103, diagnosis kit 100 disposed to carrier table 119 is rotated, as described previously, so that the sample liquid can be introduced from chamber 102 to channel 103 more efficiently by a centrifugal force.

Diagnosis kit 100 is fixed to carrier table 119 is rotated to uniformly distribute the red blood cells into cavities 115. At this moment, the manipulations of changing the rotating speed and reversing the direction of the rotation may be executed. Unnecessary portion of the red-blood-cell adjusted liquid is moved to chamber 105 connected with test plates 104 on diagnosis plates 101 and collected as waste liquid by continuing the rotation even after the red blood cells are introduced into cavities 115. Thus, the centrifugal force is utilized to move the red-blood-cell adjusted liquid more efficiently from liquid reservoir 113 to test plates 104 and further to cavities 115 from test plates 104.

A method of measuring the fluorescence radiated from diagnosis kit 100 will be described below.

Cavities 115 in test plates 104 can be tested entirely and efficiently by a pick-up method used for CD or DVD. This method can utilize a currently-available optical pick-up device, thus allowing the device to be manufactured to have a small size at low cost. The pick-up method will be described below.

Laser module 116 is fixed to laser module moving unit 121. Laser module moving unit 121 is moved from side to side by laser module drive unit 122, so that laser module 116 can be moved along straight line 116c that passes through center axis 100c of diagnosis kit 100.

A screw, for instance, can be used as laser module moving unit 121, and a stepping motor can be used as laser module drive unit 122.

Photo detector 117 including fluorescence filter 118 is fixed to photo detector moving unit 123. Photo detector moving unit 123 is moved from side to side by photo detector drive unit 124, so that photo detector 117 can be moved along straight line 117c that passes center axis 100c of diagnosis kit 100.

A screw, for instance, can be used as photo detector moving unit 123, and a stepping motor, for instance, can be used as photo detector drive unit 124.

Both laser module 116 and photo detector 117 are moved along straight lines 116c and 117c in synchronization with each other, thereby allowing photo detector 117 to measure one after another cavities 115 aligned in a row directly below laser module 116. Here, the expression “aligned in a row” means one row that is in parallel to a range along straight line 116c in which laser module 116 is moved.

Upon completing the measurement of cavities 115 aligned in the first row, carrier table 119 is rotated by carrier table drive unit 120. Diagnosis kit 100 is thus rotated by rotating carrier table 119, and examines cavities 115 sequentially one by one aligned in the next row which is not measured. All of cavities 115 can be measured in this manner by repeating the rotation of carrier table 119 and the measurement of the cavities 115 aligned in each row.

Control unit 125 can execute a series of operation control of carrier table drive unit 120, laser module drive unit 122, and photo detector drive unit 124, as well as intensity adjustment and focal point control of the excitation light from laser module 116.

Control unit 125 includes a built-in control circuit for taking in signals of luminescence as being a result of measurement data captured by photo detector 117, and these signals are received in system device 126 which can perform data processing and data analysis.

System device 126 can execute a total control of the drive units and the operation units in this measuring system.

Judgment can be made from the result detected in this manner as to whether or not the red blood cells in the biological specimen are parasitized with extraneous organisms and infected with disease.

In the above method, the specific excitation light is applied from above diagnosis kit 100, and luminescence, if occurs in any of cavities 115, is detected with photo detector 117 disposed under diagnosis kit 100. Alternatively, the specific excitation light is applied from below diagnosis kit 100, i.e., from surface 104b. In this case, the luminescence produced in cavity 115 is reflected, and intensity of the reflected light is measured by using a half mirror.

When the above detection method is used, the biological specimen can be irradiated easily with the excitation light and is detected at high sensibility even if the biological specimen is not spread out into a single layer inside cavities 115, since the excitation light is irradiated from below diagnosis kit 100.

Besides, the intensity of luminescence can be detected while the excitation light is turned off, as another detection method, to avoid the excitation light from entering photo detector 117.

To turn off the excitation light in this case, a pulsed light that illuminates only for a specified duration can be used. Alternatively, a shutter may be provided in an optical path of the excitation light.

The above detection method can reduce adverse influences of scattered light caused by irradiation of the excitation light to objects other than the targeted luminescent substance and the transmitted beam not absorbed by the luminescent substance without even using fluorescence filter 118, thereby providing measurement of the fluorescence originating only from the luminescent substance with a high sensibility.

An intensity of the fluorescence can be detected immediately after the excitation light is turned off, and it is necessary to detect the intensity of the fluorescence while the luminescent substance stays illuminant after being excited.

A lighting time of the excitation light and detection timing can be adjusted easily with a lock-in amplifier.

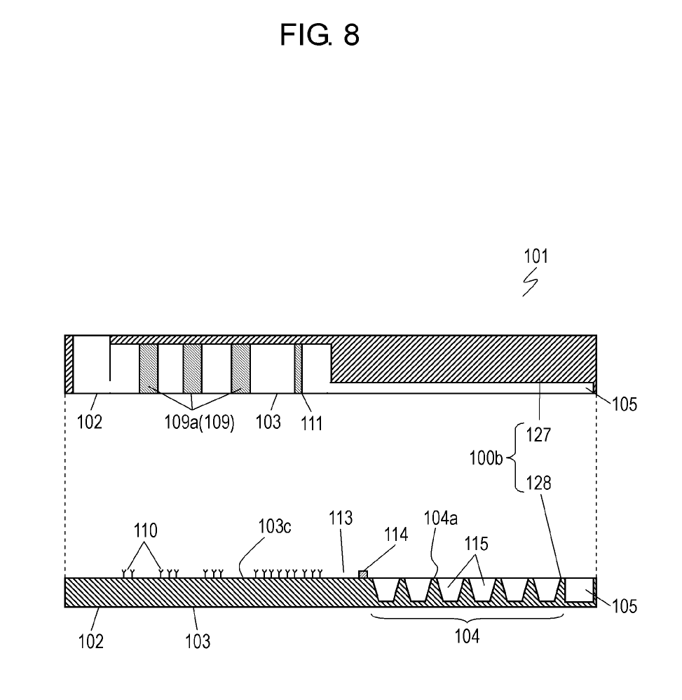

A method of manufacturing diagnosis kit 100 will be described below. FIG. 8 is a sectional view of diagnosis kit 100 for illustrating the method of manufacturing diagnosis kit 100.

Base plate 100b for diagnosis plate 101 includes upper substrate 127 and lower substrate 128 that are joined to each other. Upper substrate 127 constitutes cavity 115 excluding a bottom surface, channel 103 excluding bottom 103c, an upper surface of test plate 104, and a part of cavity 115. The materials listed above as being suitable to form test plate 104 can also be used as a material of upper substrate 127. When fibrous materials 109a are formed as non-target substance trap 109, a substrate mainly containing silicon is used as a material to make upper substrate 127. Since fibrous materials 109a are made mainly of silicon, fibrous materials 109a joined directly with upper substrate 127 can be formed when the surface of upper substrate 127 is made of silicon.

Lower substrate 128 constitutes a bottom surface of each of cavities 115, bottom 103c of channel 103, liquid reservoir 113, test plate 104, cavities 115, and a part of each of cavities 115. Any of materials suitable for upper substrate 127 and test plate 104 can also be used as a material of lower substrate 128. The same material as test plate 104 is preferably used so that lower substrate 128 and test plate 104 can be molded in one piece.

Non-target substance capturing probes 110 are preferably formed on lower substrate 128 since capturing probes 110 formed on the bottom surface of channel 103 can function more accurately and therefore desirable.

When non-target substance trap 109 of a porous material and permeable plate 111 having slits 112 are used as target-substance path 108, they can be joined to one of upper substrate 127 and lower substrate 128 before the two substrates are joined together.

In diagnosis kit 100 according to Embodiment 1 shown in FIG. 8, the walls of diagnosis plate 101, such as the side surfaces of chamber 102 and channel 103, are formed by upper substrate 127, but may be formed by lower substrate 128 instead.

Upper substrate 127 and lower substrate 128 constructed as illustrated are joined together. In method of joining, the individual joining portions of the upper substrate 127 and lower substrate 128 are joined precisely by using an alignment joining machine after their surface are activated by an ashing process using, e.g. O₂ plasma. Here, any of excimer laser and ozone plasma may be used for activation of the surfaces.

The above manufacturing method is just an example, and is not limited to.

Diagnosis kit 100 according to Embodiment 1 can detect extraneous organisms in the red blood cells quickly and easily from a small amount of biological specimen by using single diagnosis kit 100.

In addition, channel 103 capable of extracting red blood can extract the red blood cells containing infected blood cells that are specific target cells, from a biological specimen containing cells. The extraneous organisms in the red blood cells can be detected at high sensibility since only the extracted red blood cells exist in test plate 104.

In other words, substance stained in the process of staining the nucleic acid is not only the infected blood cells but also the nucleic acid of the white blood cells originally existing in the biological specimen. Since the stained white blood cells are held within channel 103, only the red blood cells are extracted into test plate 104, thereby preventing the white blood cells from erroneously examined during measurement of the luminescence, and improving the detection accuracy.

It thus becomes possible to determine the onset of infectious disease, such as malaria infection, even in a stage where no subjective symptoms are present by virtue of achieving the highly accurate measurement by using the red blood cells extracted accurately as mentioned, so as to contribute to early diagnosis of the infectious diseases.

When centrifugal separation is used to separate red blood cells from whole blood, a larger amount of whole blood be necessary than the amount actually needed for the examination, or the number of preparation processes becomes increased in order to dilute the whole blood with a solvent to increase the amount before the centrifugal separation. Diagnosis kit 100 according to Embodiment 1 does not require a large amount of sample blood since it does not take a large-scale device like the centrifugal separator. The measurement can be made easily in a short period of time by using only red blood cells with simple steps of collecting only about one microliter of sample blood from a fingertip etc. and injecting it into chamber 102.

Furthermore, diagnosis kit 100 is not only suitable for simple tests in a home, airport, seaport and clinical facility, but also useful in any region where the examination using red blood cells is needed to help diagnose easily especially for diseases such as malaria, since it requires no preparation process of staining, diagnosis can be made only with a biological specimen, and it does not require a large-scale expensive device such as a centrifugal separator.

In addition, diagnosis kit 100 using the diagnostic method according to Embodiment 1 is easy to handle without a complex manipulation.

According to Embodiment 1, diagnosis kit 100 is used for the diagnostic purpose of diseases related to red blood cells. Diagnosis kit 100 can be used for other purposes, such as DNA and protein examinations.

### Exemplary Embodiment 2

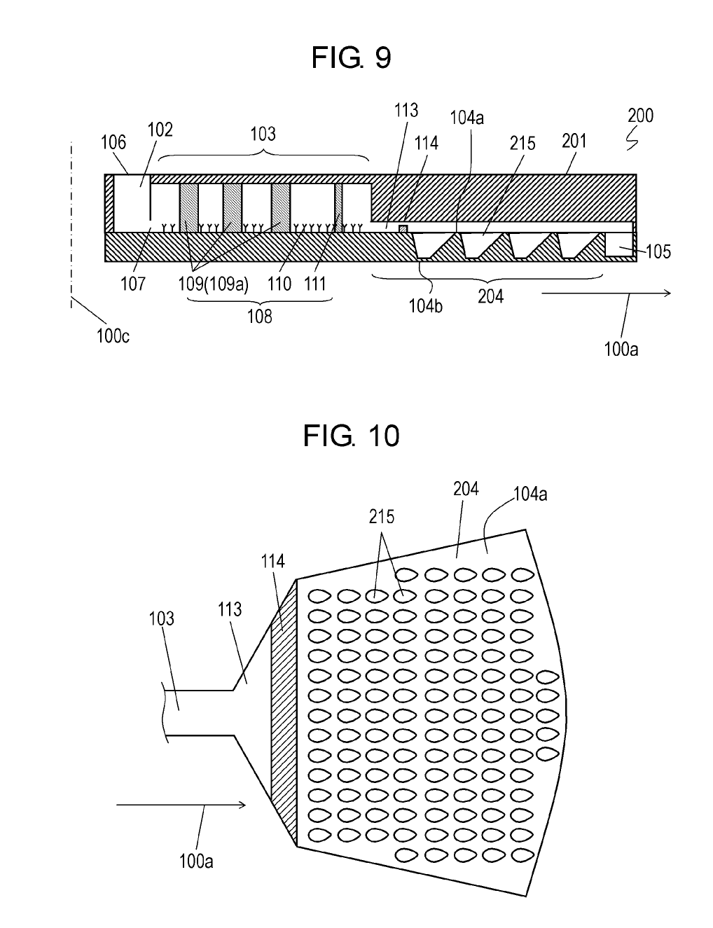

FIG. 9 is a sectional view of diagnosis kit 200 according to Exemplary Embodiment 2. In FIG. 9, components identical to those of diagnosis kit 100 according to Embodiment 1 shown from FIGS. 1 to 8 are denoted by the same reference numerals. Diagnosis kit 200 according to Embodiment 2 includes diagnosis plate 201 and test plate 204 instead of diagnosis plate 101 and test plate 104 of diagnosis kit 100 according to Embodiment 1. Test plate 204 has cavities 215 instead of cavities 115 of test plate 104 according to Embodiment 1. Cavities 215 have shapes different from cavities 115.

FIG. 10 is a top view of test plate 204. Cavities 215 are provided in surface 104a of test plate 204, similarly to test plate 104 according to Embodiment 1.

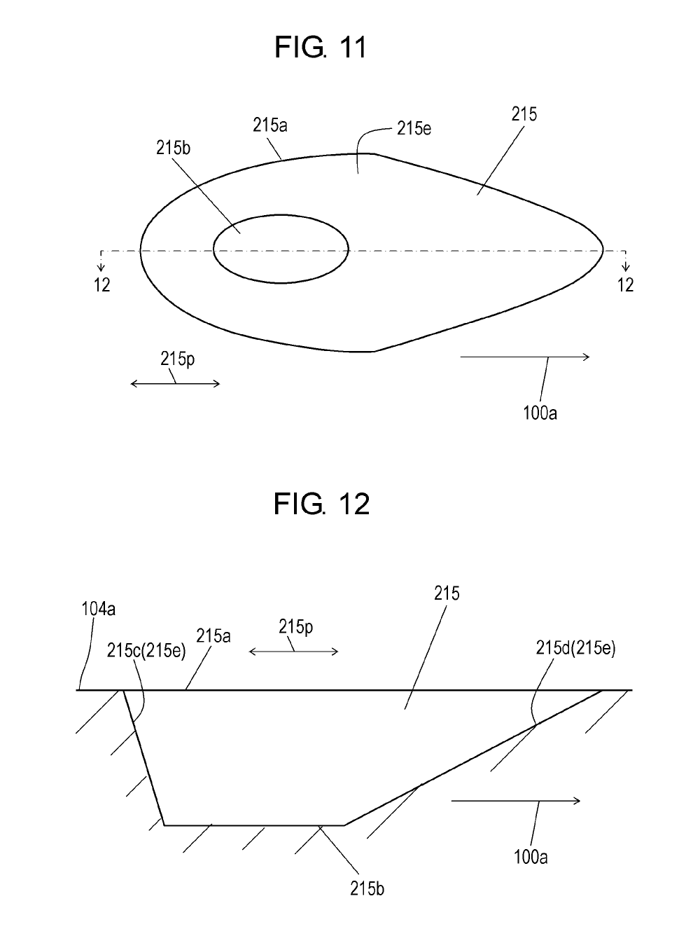

FIG. 11 is a top view of cavity 215. FIG. 12 is a sectional view of cavity 215 at line 12-12 shown in FIG. 11. Cavity 215 has opening 215a opened to surface 104a of test plate 104, bottom surface 215b, and inner wall 215e extending from bottom surface 215b to opening 215a. Bottom surface 215b is flat.

Cavity 215 has a conical shape flaring from bottom surface 215b toward opening 215a. Opening 215a of cavity 215 can have a circular or rectangular shape. Opening 215a of cavity 215 preferably has a shape elongating in longitudinal direction 215p parallel to direction 100a. The shape of opening 215a may be oval. Opening 215a may preferably have an egg shape, as shown in FIG. 11, having a width in a direction perpendicular to longitudinal direction 215p at one end of direction 215 is larger than a width in the direction perpendicular to longitudinal direction 215p at another end opposite to direction 215.

Inner wall 215e of cavity 215 is not perpendicular to surface 104a and inclines with respect to surface 104a, as shown in FIG. 12, for expelling an excessive portion of the biological specimen by the centrifugal force. The slope of portion 215d of inner wall 215e in direction 100a is gentler than the slope of portion 215c of inner wall 215e in the direction opposite to direction 100a. Portion 215c of inner wall 215e is located in the direction directed to center axis 100c, which is an inner circumferential when diagnosis kit 200 rotates about center axis 100c. Portion 215d of inner wall 215e is located in the direction directed to an outer circumferential when diagnosis kit 200 rotates about center axis 100c. This structure allows an excessive portion of the biological specimen to be expelled more efficiently toward chamber 105 by the centrifugal force.

It may be erroneously detected that extraneous organisms exist the red blood cells, thus not detecting accurately since the biological specimen is distributed excessively into cavities 115 due to cleaning which causes a difference in density of the biological specimen among the cavities. Cavities 215 having the above shape enable the biological specimen to be distributed easily into single layer.

Therefore, it is not necessary to inject additional chemical solution, such as a buffer, culture medium, surface-active agent, or enzymes for washing out into diagnosis kit 100.

Besides, diagnosis plate 201 and the diagnosis kit are easily manufactured since diagnosis kit 200 may not necessarily include a complex structure for injecting such chemical solution.

In the case that diagnosis kit 200 is fixed to an upper surface of rotatable carrier table 119 (FIG. 7), diagnosis kit 200 may be required to remove temporarily from carrier table 119 for cleaning with a chemical solution. Cavities 215 of diagnosis kit 200 according to Embodiment 2 are configured to move an excessive portion of the biological specimen as waste liquid from cavities 215 to chamber 105 by the centrifugal force, thereby enabling the biological specimen inside cavities 215 to spread out into a single layer.

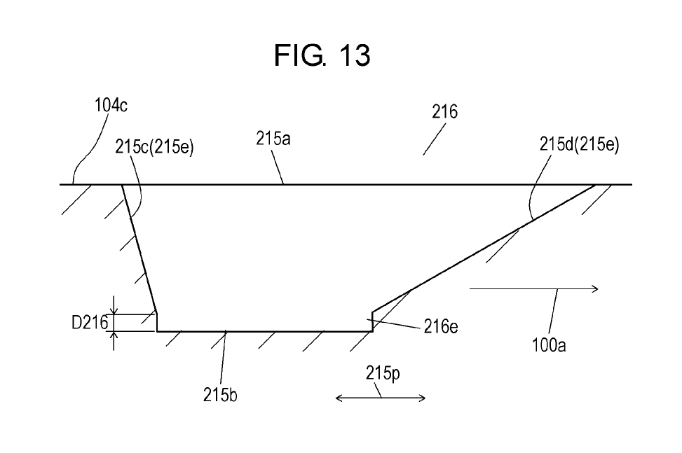

FIG. 13 is a sectional view of another cavity 216 of diagnosis kit 200 according to Embodiment 2. In FIG. 13, components identical to those of cavity 215 shown in FIG. 12 are denoted by the same reference numerals. Cavity 216 has recess 216e having a flat bottom and extending toward opening 215a while maintaining a constant sectional area from bottom surface 215b. Inner wall 215e extends from an edge of recess 216e to opening 215a. Depth D216 of recess 216e preferably ranges from 3 μm to 10 μm to enable the target substance of red blood cells to spread out into a single layer. If depth D216 is larger than 10 μm, an excessive amount of the target substance may remain inside cavity 216, and accumulate into multiple layers. This structure can make the biological specimen spread into a single layer of uniform density by the centrifugal force without the need of cleaning, thereby making the examination of the target substance simple and efficient.

When diagnosis kit 200 has a circular shape, cavities 215 (216) of test plate 204 are preferably arranged in conformity with the outer periphery of base plate 101b.

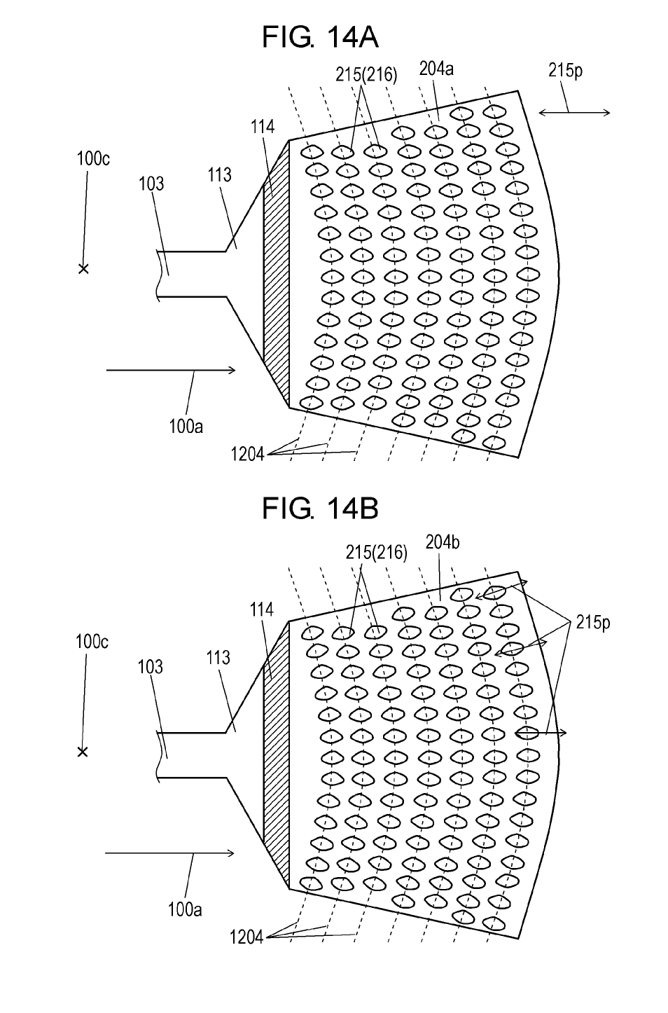

FIG. 14A is a top view of another test plate 204a of diagnosis kit 200 according to Embodiment 2. In FIG. 14A, components identical to those of test plate 204 shown in FIG. 10 are denoted by the same reference numerals. In test plate 204a, cavities 215 (216) are arranged along concentric circles 1204 about center axis 100c of base plate 101b. This configuration facilitates examining the target substance in each of cavities 215 (216) more efficiently in a short time.

FIG. 14B is a top view of still another test plate 204b of diagnosis kit 200 according to Embodiment 2. In FIG. 14B, components identical to those of test plate 204a shown in FIG. 14A are denoted by the same reference numerals. In test plate 204b, cavities 215 (216) are arranged along concentric circles 1204 about center axis 100c of base plate 101b. In test plate 204a shown in FIG. 14A, longitudinal directions 215p of cavities 215 (216) are parallel with direction 100a. In test plate 104 shown in FIG. 14B, longitudinal directions 215p of cavities 215 (216) are directed to center axis 100c. This configuration can facilitate examination of the target substance in each of cavities 215 (216) even more efficiently in a short time.

### Exemplary Embodiment 3

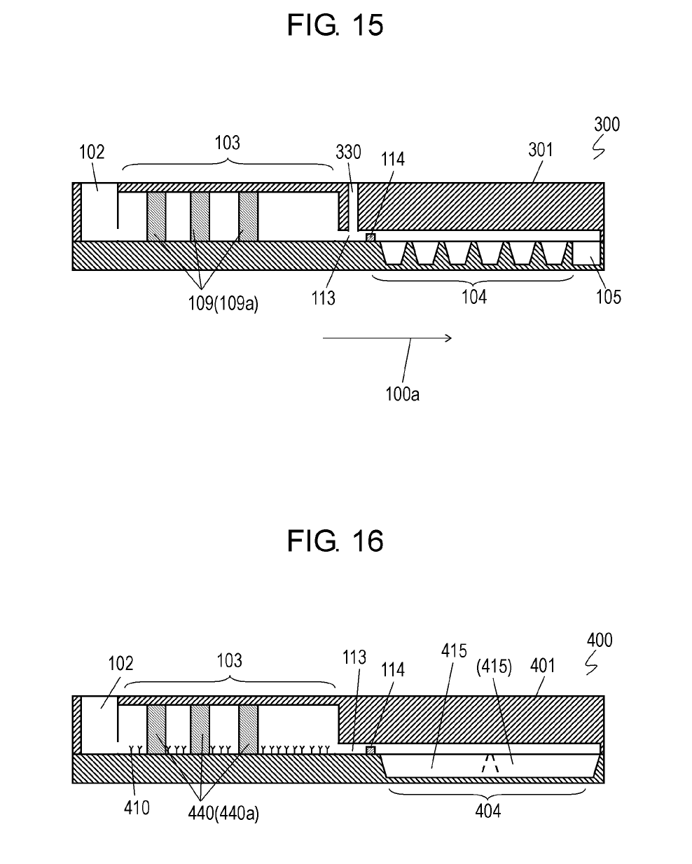

FIG. 15 is a sectional view of diagnosis plate 301 of diagnosis kit 300 according to Exemplary Embodiment 3. In FIG. 15, components identical to those of diagnosis kit 100 according to Embodiment 1 shown from FIGS. 1 to 8 are denoted by the same reference numerals. Diagnosis kit 300 includes diagnosis plate 301 instead of diagnosis plate 101 according to Embodiment 1. Diagnosis kit 300 according to Embodiment 3 can extract white blood cells from blood and living tissue derived components, and analyze the white blood cells using test plate 104.

In diagnosis kit 300, diagnosis plate 301 has through-hole 330 formed therein between channel 103 and test plate 104 to provide a path to allow channel 103 to communicate with an outside of diagnosis kit 300. Through-hole 330 is located between channel 103 and test plate 104. In each of diagnosis plates 301 of diagnosis kit 300, through-hole 330 extends from liquid reservoir 113 in an upward direction perpendicular to direction 100a. Diagnosis kit 300 does not include non-target substance capturing probe 110 and permeable plate 111 for trapping white blood cells which are included in the kit according to Embodiment 1.

An examination method with diagnosis kit 300 will be described below. First, red blood cells are introduced into test plate 104 similarly to the kit according to Embodiment 1, and cause non-target substance trap 109 to capture white blood cells. Then, the red blood cells on test plate 104 are washed out by injecting a cleaning liquid from through-hole 330 into liquid reservoir 113.

Then, the white blood cells trapped in channel 103 are forced to move into test plate 104 by introducing a chemical substance, e.g. a catabolic enzyme, such as Trypsin or Accutase®, capable of removing the trapped white blood cells into channel 103 from chamber 102. Then, the white blood cells can be examined in test plate 104 similarly to the red blood cells. The chemical substance can keep shapes of the white blood cells, thus reducing damages to the white blood cell membranes. Regardless of the above, the white blood cells may be removed from non-target substance trap 109 by breaking the white blood cell membranes.

Sample specimens made of blood or living tissue derived components may contain circulative tumor cells (hereinafter CTC). Diagnosis kit 300 according to Embodiment 3 is also capable of detecting the CTC.

In this case, the CTC is stained first with a fluorescence-marked specific antibody inside chamber 102. The white blood cells and CTC are then trapped with non-target substance trap 109 in channel 103 to have the red blood cell components extracted from channel 103.

White blood cells and CTC tends to be adsorbed on surfaces of fibrous materials 109a since the white blood cells and CTC have much more admolecules attached onto surfaces than red blood cells to adhering to foreign bodies and other cells in blood and other tissue cells. Since fibrous materials 109a have large surface areas, fibrous materials 109a can capture a large amount of cells, such as the white blood cells and CTC, having adherence.

Fibrous materials 109a may additionally be coated with antibodies on surfaces thereof previously for securely capturing the CTC. As a result, the CTC can be chemically adsorbed more reliably.

All of the red blood cells extracted from channel 103 pass through or are washed out from test plate 104, and discharged into chamber 105 for collecting waste fluid. The red blood cells on test plate 104 can be washed out by injecting a cleaning liquid from through-hole 330.

Then, the white blood cells and CTC in channel 103 are forced to move to test plate 104 by using a chemical substance capable of removing the white blood cells and CTC trapped in channel 103. It is examined whether or not the CTC exists by measuring the fluorescence. Due to the capability of detecting the CTC in the specimen made of a small amount of blood or living tissue derived components at the early stage, the CTC can be detected and the medical treatment be given before the CTC reaches and infiltrates (metastasizes) other tissues. In addition, diagnosis kit 300 is useful for prognosis, pin-pointing a primary region, and determination of the therapeutic effect of anticancer agents, by separating the CTC and performing genome analysis.

According to Embodiment 3, the capillary action can also be used when injecting the specimen into chamber 102.

### Exemplary Embodiment 4

FIG. 16 is a sectional view of diagnosis plate 401 of diagnosis kit 400 according to Exemplary Embodiment 4. In FIG. 16, components identical to those of diagnosis kit 100 according to Embodiment 1 shown from FIGS. 1 to 8 are denoted by the same reference numerals. Diagnosis kit 400 includes diagnosis plate 401 instead of diagnosis plate 101 of diagnosis kit 100 according to Embodiment 1. Diagnosis kit 400 according to Embodiment 4 is configured to analyze blood plasma extracted from blood and living tissue derived components.

Diagnosis plate 401 includes test plate 404, non-target substance traps 440, and non-target substance capturing probes 410 instead of test plate 104, non-target substance traps 109, and non-target substance capturing probes 110 of diagnosis plate 101 according to Embodiment 1. Non-target substance traps 440 are disposed in channel 103. Non-target substance capturing probes 410 is disposed in channel 103. Non-target substance capturing probes 410 are bonded specifically with white blood cells and red blood cells. Inside channel 103, non-target substance traps 440 and non-target substance capturing probes 410 capture only blood cell components, such as red blood cells and white blood cells, from the sample liquid stored in chamber 102 to extract the blood plasma. The blood plasma is then examined in test plate 404.

Non-target substance traps 440 disposed in channel 103 capture only the blood cells from the sample liquid. Each of non-target substance traps 440 is made of, for instance, plural fibrous materials 440a similar to fibrous materials 109a according to Embodiment 1.

The shortest distance of each space between adjoining fibrous materials 440a is smaller than the substances, blood corpuscles, such as red blood cells and white blood cells, to be trapped. Non-target substance traps 440 are made of mutually entangled fibrous materials 440a. Among solutes contained in the biological specimen, blood cell components, such as the red blood cells and the white blood cells, and other substances having the largest diameter greater than the spaces among fibrous materials 440a are captured by fibrous materials 440a as the substances to be trapped. On the other hand, the blood plasma capable of passing through the spaces among fibrous materials 440a can pass through fibrous materials 440a, thereby enabling non-target substance traps 440 to extract the blood plasma by allowing the blood plasma to pass through non-target substance traps 440.

To extract the blood cells, the spaces among fibrous materials 440a of non-target substance traps 440 are controlled to preferably range from about 1 μm to 3 μm.

The blood-plasma components extracted in channel 103 are moved to test plate 404 where the blood-plasma components are distributed into cavities 415 formed in test plate 404.

The blood plasma in the blood contains water, electrolyte, glucose, lipid, waste matter, and plasma proteins. Among these components, the plasma proteins include albumins, globulins, fibrinogen and the like, and globulins in particular are closely related to the immune function and the allergic reaction.

An increase of immunoglobulin causes hyperproteinemia which leads to conditions, such as pycnosis of blood, chronic infections, collagen disease, and autoimmune disease due to dehydration. On the other hand, a decrease of albumins causes hypoalbuminemia which leads to conditions, such as under-nutrition and transudation of proteins attributed to exsanguinations. When there is a decrease in the yield of immunoglobulin in the blood plasma, it leads to a condition to get infectious diseases. These symptoms can be diagnosed by measuring an amount of plasma proteins in the blood plasma.

Diagnosis plate 401 may include an electrode disposed inside cavity 415 of test plate 404. The glucose in the blood plasma can be tested by pre-coating enzyme, such as glucose oxidase, in cavity 415. A glucose concentration can be measured electrically or colorimetrically from hydrogen peroxide produced by the reaction of this enzyme (glucose oxidase) with the glucose in the blood.

Test plate 404 may have cavities 415 therein. Diagnosis plate 401 may have probes, secured individually in cavities 415. This structure can examine plasma components, and preferably performs various kinds of biochemical analysis at once.

According to Embodiment 4, the capillary action can be used when injecting the specimen into chamber 102.

### Exemplary Embodiment 5

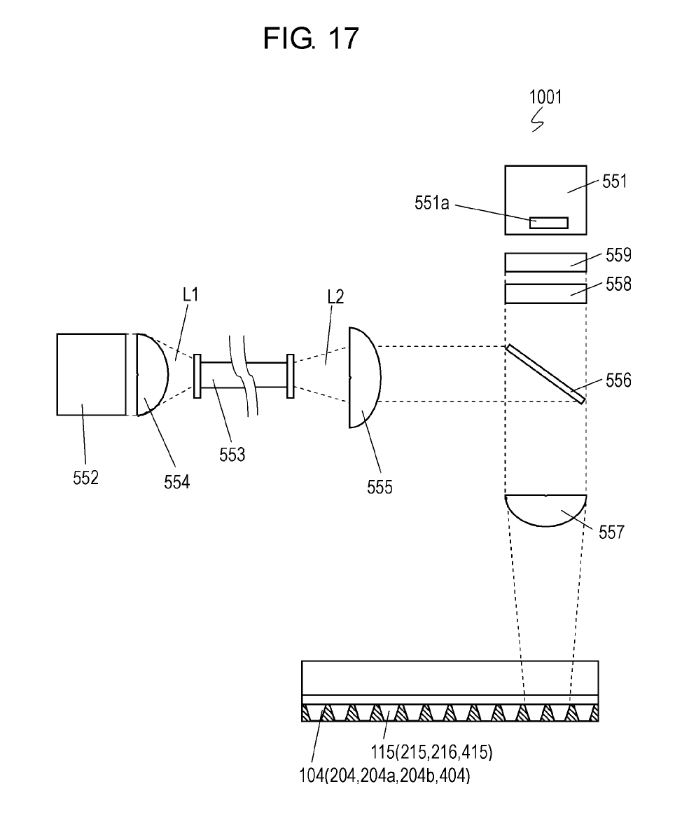

FIG. 17 is a schematic diagram of detecting apparatus 1001 according to Exemplary Embodiment 5. In FIG. 17, components identical to those of diagnosis kits 100 to 400 according to Embodiments 1 to 4 shown in FIGS. 1 to 16 are denoted by the same reference numerals. Detecting apparatus 1001 includes camera 551 for detecting fluorescence emitted from test plate 104 (204, 204a, 204b or 404).

Detecting apparatus 1001 can radiate laser light L2 uniformly to test plate 104 (204, 204a, 204b or 404) or to cavities 115 (215, 216 or 415) in test plate 104 (204, 204a, 204b or 404) by using optical fiber 553. Laser light L1 emitted from laser source 552 enters optical fiber 553, and transmits inside a core of optical fiber 553 while reflecting on a core wall. As a result, the laser profile becomes nearly uniform. After having adjusted a spreading angle of laser light L2 exited from optical fiber 553 with lens 555, laser light L2 is reflected by mirror 556 capable of reflecting only a specific band of wavelengths including the wavelength of laser light L2. The reflected laser light L2 is radiated to cavities 115 (215, 216 or 415) of test plate 104 (204, 204a, 204b or 404) through object lens 557.

Test plate 104 (204, 204a, 204b or 404) is a flat plate used for detecting fluorescence, and a specimen can be disposed in cavities 115 (215, 216 or 415). The shape of test plate 104 (204, 204a, 204b or 404) may be discoidal, tabular, polygonal and the like. Test plate 104 (204, 204a, 204b or 404) may not necessarily have cavities. In this case, the specimen may be tested by spotting the specimen on surface 104a.

When detecting whether or not extraneous organisms exist in red blood cells, for example, red blood cells soaked in a stain solution are disposed in cavities 115 (215, 216 or 415) of test plate 104 (204, 204a, 204b or 404). Normal red blood cells are not stained with the stain solution since they do not have nucleuses, and they therefore do not make luminous emission (fluorescence) even when irradiated with light of the specific wavelength. If the red blood cells are infected with parasitic extraneous organisms, the red blood cells parasitized with the extraneous organisms produce fluorescence when irradiated with the laser light since the nucleuses derived from the extraneous organisms have been stained.

A portion of the fluorescence that passes through object lens 557 forms a parallel light which enters and passes through mirror 556. The fluorescence that passing through mirror 556 forms an image on image sensor 551a, such as a CCD element in camera 551, by way of an image-forming lens, and the image is hence detected. The red blood cells can be diagnosed by processing the detected image.

The wavelength of laser light L1 may be selected according to an excitation wavelength of a reagent used for the examination.

Lens 554 is disposed between laser source 552 and optical fiber 553 to efficiently introduce laser light L1 into optical fiber 553. The efficiency of energy is improved by disposing lens 554 to an appropriate position. It is more preferable to use lens 554 having a property guiding the entire laser light L1 into optical fiber 553.

Lens 555 forms laser light L2 having substantially the same diameter as a pupil diameter of object lens 557. Distribution of intensity of the excitation light radiated to test plate 104 can be made uniform by disposing lens 555 to an appropriate position.

Although either a dichroic mirror or a half mirror can be used, for instance, as mirror 556, the dichroic mirror is more preferable since it is capable of reflecting a specific wavelength and passing fluorescence efficiently.

Fluorescence filter 558 capable of blocking light other than the fluorescence may be preferably disposed between mirror 556 and image-forming lens 559.

Detecting apparatus 1001 according to Embodiment 5 can cover a surface as a single unit of the detection by making use of camera 551. For example, a surface of about 1.3 mm by 1 mm can be detected at a time when an object lens having a magnification of 5 times and ½-inch image sensor 551a are used. The diagnosis can hence be made within a short time.

Test plate 104 (204, 204a, 204b or 404) is at rest during the detection. If test plate 104 (204, 204a, 204b or 404) is larger than a unit size of the image detection surface, test plate 104 (204, 204a, 204b or 404) may be rotated or otherwise moved to detect one area after completing the detection of another area.

Since the image detection is carried out by using camera 551, the specimen can be distinguished from other substances by having the image processed. For instance, white blood cells and substances, such as foreign objects, other than the specimen adhering to the test plate emit fluorescence when detecting whether or not extraneous organisms as the specimen exist in red blood cells by using a diagnosis kit which does not include a non-target substance trap according to Embodiment 1. However, the red blood cells can be distinguished from the white blood cells and the other foreign objects by having their images processed since they are different in sizes, brightness, shapes and the like from each other.

When test plate 104 (204, 204a, 204b or 404) is irradiated with laser light L1 as it is radiated from laser source 552, variations may occur in the intensity within the surface being irradiated. The variations in the intensity of irradiation within the surface tend to cause variations in intensity of excitation in the same surface. In the case of detecting that extraneous organisms as the specimen exist in red blood cells on test plate 104 (204, 204a, 204b or 404) where other fluorescent substances, such as white blood cells and foreign objects other than the specimen adhering to the test plate, coexist, the specimen can be identified by the brightness obtained from the image processing since the specimen normally exhibits fluorescence at a weaker intensity. However, when the intensity of excitation has a variation within the surface being irradiated, and if the specimen exists in an area where the excitation intensity is strong, and the substances other than the specimen are in an area where the excitation intensity is weak, their fluorescence intensities become generally equal, hence hardly distinguishing by the image processing.

In detecting apparatus 1001 according to Embodiment 5, the intensity distribution of the excitation light radiated to test plate 104 (204, 204a, 204b or 404) can be leveled uniform by introducing laser light L2 into optical fiber 553 before irradiating test plate 104. Since this facilitates processing the image properly, the diagnosis can be made accurately.

The core of optical fiber 553 preferably has a cross section having a rectangular shape which promotes the light more uniform than a circular shape.

The length of optical fiber 553 preferably ranges from 3 m to 10 m. Detecting apparatus 1001 becomes bulkier and heavier as a whole if optical fiber 553 is too long, whereas the uniformity of the light in the surface being irradiated becomes insufficient if too short.

Other methods are also available besides optical fiber 553 to improve uniformity of the intensity distribution of the excitation light radiated to test plate 104, such as a structure having a combination of multiple lenses along a traveling direction of the laser light, a structure using a fly-eye lens, and a structure using a diffractive optical element (DOE).

In detecting apparatus 1001 according to Embodiment 5, the efficiency of energy use is improved by irradiating test plate 104 with laser light L2 uniformalized by optical fiber 553 than other apparatuses equipped with an LED lamp or a mercury lamp.

### Exemplary Embodiment 6

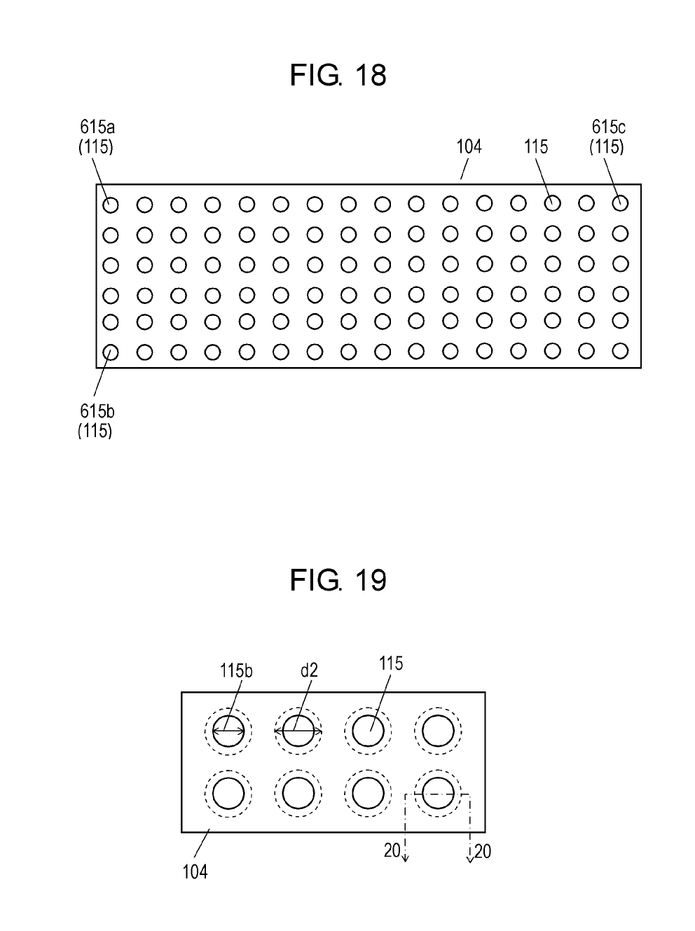

FIG. 18 and FIG. 19 are a top view and an enlarged view of test plate 104 according to Exemplary Embodiment 6, respectively. In FIGS. 18 and 19, components identical to those of diagnosis kits 100 to 400 and detecting apparatus 1001 according to Embodiments 1 to 5 shown in FIGS. 1 to 17 are denoted by the same reference numerals. A detecting apparatus according to Embodiment 6 has a function of detecting positions of cavities 115 in test plate 104 when fluorescence emitted from test plate 104 having cavities 115 are detected with camera 551 similarly to detecting apparatus 1001 according to Embodiment 5.

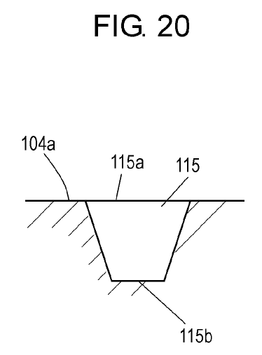

FIG. 20 is a sectional view of cavity 115 at line 20-20 shown in FIG. 19. Fluorescence is preferably detected with camera 551 focused on bottom surface 115b of cavity 115.

If a depth of cavity 115 is larger than a focal depth of object lens 557, camera 551 is focused on the vicinity of bottom surface 115b of cavity 15 when detecting a specimen existing on bottom surface 115b of cavity 15. Cavity 115 may have a tapered shape that flaring from bottom surface 115b toward surface 104a of test plate 104. In this case, fluorescence from a fluorescent source may possibly be detected blurred due to the lens shifted out of focus if there are fluorescent sources on surfaces, such as a side surface of cavity 115 and surface 104a of test plate 104, other than bottom surface 115b. In this case, an accuracy of the detection may be impaired due to the difficulty of identifying the specimen to be detected primarily with an image processing.

According to Embodiment 6, test plate 104 includes a recognition pattern containing at least three points at predetermined portions. The recognition pattern is constituted by parts of cavities 115. The recognition pattern may constituted by cavities 615a, 615b and 616c out of cavities 115 provided at very edges of test plate 104, as shown in FIG. 18. The recognition pattern may be constituted by three or more marks located at the edges on surface 104a of test plate 104. These marks can be circles or crisscross, and formed by printing, etching or molding.

An initial position of test plate 104 can be adjusted by recognition of small deviations in positions of cavities 115 and angles of cavities 115 by using the recognition pattern even if test plate 104 has small variations in the positions of cavities 115 attributed to manufacturing deviations. Affine-transformation, for instance, can be used for the recognition of such small deviations in the positions of cavities 115 and the angles of cavities 115.

After the initial position is adjusted and one part detected, the detecting operation is repeated by detecting the other parts while shifting test plate 104 little by little according to a field range of the camera. Shifting of test plate 104 includes movement and rotation of the test plate placed on an automated stage.

While shifting test plate 104, the detecting positions may shift from the original positions of cavities 115 due to factors, such as variations in spaces among cavities 115 in test plate 104, variations in sizes of cavities 115, variations in shapes of cavities 115, and variations in control accuracy of a driving source for moving and rotating the automated stage. According to Embodiment 6, camera 551 detects area d2 which includes bottom surface 115b of actual cavity 115 and which is larger than bottom surface 115b, as shown in FIG. 19. Area d2 has a diameter of about 1.3 to 1.5 times that of bottom surface 115b of cavity 115. Accordingly, camera 551 can detect the target substance inside cavity 115 even after the detecting operation is repeated while moving test plate 104.

As discussed, the above configuration can facilitate diagnosis of the target substance in a short time since it is designed to recognize and detect only certain parts of cavities 115 of test plate 104 instead of detecting test plate 104 entirely.

### INDUSTRIAL APPLICABILITY

A diagnosis kit according to the present invention can detect extraneous organisms in red blood cells easily with a small amount of biological specimen, and is expected to use for diagnosing diseases related to the red blood cells.

### REFERENCE MARKS IN THE DRAWINGS

100 Diagnosis Kit
100b Base Plate
101 Diagnosis Plate
102 Chamber (First Chamber)
103 Channel
104 Test Plate
105 Chamber (Second Chamber)
108 Target-Substance Path
109 Non-Target Substance Trap
109a Fibrous Material
110 Non-Target Substance Capturing Probe
111 Permeable Plate
112 Slit
115 Cavity
117 Photo Detector
118 Fluorescence Filter
215 Cavity
216 Cavity

1. A diagnosis kit configured to detect whether or not an extraneous organism exists in a red blood cell by using a biological specimen containing the red blood cell, and a stain solution capable of staining nucleic acid, the kit comprising:
a base plate;
at least one diagnosis plate disposed at the base plate, the at least one diagnosis plate including
a first chamber configured to store the stain solution and to have the biological specimen injected into the stain solution, the first chamber having a wall surface having a through-hole provided therein,
a channel having one end-hole and another end opposite to the one end, the one end of the channel being connected to the first chamber via the through-hole, the channel being configured to extract the red blood cell from the biological specimen and the stain solution, and
a test plate connected to the another end of the channel, and configured to receive the extracted red blood cell; and
a second chamber provided in the base plate, and connected with the test plate for collecting a part of the biological specimen and a part of the stain solution.

2. The diagnosis kit according to claim 1,
wherein the at least one diagnosis plate comprises a plurality of diagnosis plates, and
wherein the second chamber is connected with the test plate of each of the plurality of diagnosis plates for collecting the part of the biological specimen and the part of the stain solution from each of the plurality of diagnosis plates.

3. The diagnosis kit according to claim 1, wherein a part of the through-hole has a hydrophobic treatment performed thereto.

4. The diagnosis kit according to claim 1, wherein the diagnosis plate further includes a target-substance path provided in the channel for having the red blood cell pass through the target-substance path.

5. The diagnosis kit according to claim 4, wherein the target-substance path includes a non-target substance trap having at least one of a fibrous material and a porous material, and being capable of capturing a white blood cell.

6. The diagnosis kit according to claim 5, wherein the fibrous material mainly containing silicon oxide.

7. The diagnosis kit according to claim 4, wherein the target-substance path includes a non-target substance capturing probe capable of catching a white blood cell.

8. The diagnosis kit according to claim 4, wherein the target-substance path includes a permeable plate having one or more slits provided therein.

9. The diagnosis kit according to claim 8, wherein the one or more slits elongate in a longitudinal direction, and have a length not larger than 7 μm in a direction perpendicular to the longitudinal direction.

10. The diagnosis kit according to claim 1, wherein the channel has a height not larger than 7 μm at a portion where the channel and the test plate are connected.

11. The diagnosis kit according to claim 1, wherein the diagnosis plate further includes a liquid blocking structure disposed near a joint portion where the another end of the channel and the test plate are connected, the a liquid blocking structure being configured to collect the red blood cell.

12. The diagnosis kit according to claim 1, wherein the test plate has a plurality of cavities provided therein for having the extracted red blood cell placed therein.

13. The diagnosis kit according to claim 12,
wherein the channel extends in a predetermined direction from the one end toward the another end,
wherein each of the plurality of cavities has an opening opened to an upper surface of the test plate, a flat bottom surface, and an inner wall extending from the bottom surface to the opening,
wherein each of the plurality of cavities flaring from the bottom surface toward the opening, and
wherein a slope of a portion of the inner wall in the predetermined direction is gentler than a slope of a portion of the inner wall of each of the cavities in a direction opposite to the predetermined direction.

14. The diagnosis kit according to claim 12, wherein each of the plurality of cavities has a recess provided in the bottom surface thereof, the recess having a flat bottom.

15. The diagnosis kit according to claim 14, wherein a depth of the recess is not larger than 10 μm.

16. A method of using a diagnosis kit, comprising:
preparing the diagnosis kit according to claim 1;
preparing the biological specimen and the stain solution placed in the first chamber of the diagnosis kit; and
moving the red blood cell, by a centrifugal force, from the first chamber to the test plate through the channel connected with the first chamber.

17. A method of using a diagnosis kit, comprising:
preparing the diagnosis kit according to claim 1;
preparing the biological specimen and the stain solution placed in the first chamber of the diagnosis kit; and
moving the red blood cell from the first chamber to the test plate through the channel by producing a difference in pressure between the first chamber and the channel.

18. A method of using a diagnosis kit, comprising:
preparing the diagnosis kit according to claim 1;
preparing the biological specimen and the stain solution placed in the first chamber of the diagnosis kit;
moving the red blood cell from the first chamber to the test plate through the channel;
causing the extraneous organism in the red blood cell to generate fluorescence by irradiating the moved red blood cell with light having an excitation wavelength through one of a first surface and a second surface opposite the first surface of the test plate after said moving of the red blood cell from the first chamber to the test plate through the channel; and
measuring an intensity of the fluorescence through one of the first surface and the second surface of the test plate.

19. The method according to claim 18, wherein said measuring of the intensity of the fluorescence comprises measuring the intensity of the fluorescence through a fluorescence plate capable of blocking a light having the excitation wavelength.

20. The method according to claim 18, wherein said measuring of the intensity of the fluorescence comprises measuring the intensity of the fluorescence while rotating the diagnosis kit.
